(19) United States
(12) Patent Application Publication
(10) Pub. No.: US 2022/0324809 A1
(43) Pub. Date: Oct. 13, 2022

(54) ATP-REGULATED POTASSIUM CHANNEL OPENERS AND USES THEREOF

(71) Applicant: SALZMAN GROUP LTD., Katzrin (IL)

(72) Inventors: Prakash JAGTAP, North Andover, MA (US); Andrew Lurie SALZMAN, West Tisbury, MA (US)

(21) Appl. No.: 17/753,470

(22) PCT Filed: Sep. 2, 2020
(86) PCT No.: PCT/IL2020/050953

§ 371 Date: Mar. 3, 2022

Related U.S. Application Data
(60) Provisional application No. 62/895,195, filed on Sep. 3, 2019.

(51) Int. Cl. C07D 213/82; A61P 9/12

(52) U.S. Cl. CPC C07D 213/82; A61P 9/12

(57) ABSTRACT

Compounds and compositions including an ATP-regulated potassium (K_{ATP}) channel opener can be including in pharmaceutical compositions. These compounds and compositions are useful for prevention, treatment, or management of pulmonary arterial or venous hypertension. The composition including the compounds may be formulated for intravenous, intraarterial, intramuscular, intranasal, rectal, anal, intravaginal, intratracheal, intraperitoneal, or subcutaneous, administration, or for inhalation.

### TECHNICAL FIELD

The present invention relates to compounds comprising an ATP-regulated potassium (K_{ATP}) channel opener, and to pharmaceutical compositions comprising them. These compounds and compositions are useful for prevention, treatment, or management of pulmonary arterial or venous hypertension.

Abbreviations: DCM, dichloromethane; DMF, dimethylformamide; PBS, phosphate-buffered saline; THF, tetrahydrofuran.

### BACKGROUND ART

Pulmonary hypertension (PH) is an abnormal elevation in the blood pressure of the pulmonary circulation associated with right ventricular strain and hypertrophy, muscularization of the pulmonary arteriolar bed, often leading to right heart failure and death. A variety of scenarios are known to associate with PH, including overcirculation secondary to left to right ventricular shunting, excessive vasoconstriction of the pulmonary arterioles and venules, and left heart failure. Numerous etiologies have been advanced to account for the development of PH, including failure of the shift from the fetal to newborn pulmonary circulation, ventricular septal defects, arteriovenous canal defects, excessive endothelin-1, deficient nitric oxide, pulmonary fibrosis, increased left atrial pressure secondary to mitral regurgitation or left-sided congestive heart failure, microembolism, hemoglobinopathy, chronic obstructive pulmonary disease, obstructive sleep apnea, hypoxia, high altitude, decompression sickness, sepsis, adult respiratory distress syndrome, aspiration pneumonia, neurologic adrenergic crisis, among others. It is estimated that PH affects about 1% of the global population and as many as 10% of persons older than 65 years.

Treatment of PH classically relies upon relief of the underlying diathesis and administration of vasodilators. Among the latter are endothelin-1 antagonists, nitric oxide donors, calcium channel antagonists, phosphodiesterase 5 inhibitors, prostacyclins, supplemental oxygen, hyperbaric oxygen, and extracorporeal membrane oxygenation for lung bypass.

### SUMMARY OF INVENTION

It has now been found, in accordance with the present invention, that a novel, water-soluble, vasodilating small molecule derived from the combination of the two K_{ATP} channel openers P-1075 and nicorandil, wherein the hydroxyethyl-nicotinamide fragment of nicorandil is attached to the cyanoguanidine fragment of P-1075, is an effective pulmonary-selective vasodilator. Said compound, herein identified R-1703, is intended to target the SUR2/Kir6.1 subunit of the K_{ATP} channel present in the vascular smooth muscle (VSM) of the pulmonary arteriolar bed. Activation of this channel hyperpolarizes the VSM, producing vasodilation that counters the vasoconstrictive responses to hypoxia, nitric oxide deficiency, and endothelin-1 and thereby reducing pulmonary vascular resistance.

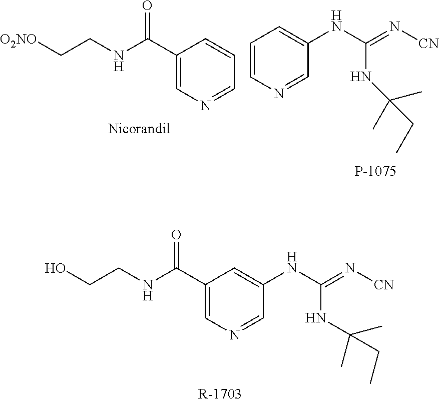

As shown herein using a well-established gold-standard rodent model of severe PH, acute intravenous (IV) administration of R-1703 eliminates high mean pulmonary arterial pressure (MPAP) without impacting peripheral blood pressure or heart rate, indicating its selectivity for the pulmonary vascular circulation at these dose levels. Compared to current pulmonary vasodilators, R-1703 is intended to be more uniformly effective, more potent, less expensive, and easier to administer.

In one aspect, the present invention thus provides a compound of the general formula I:

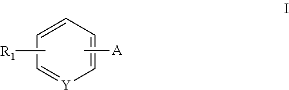

wherein Y is N, CH or N(→O),

or an enantiomer, diastereomer, racemate, or a pharmaceutically acceptable salt thereof,

wherein

A is a moiety of the formula II linked through its terminal —NH group to any carbon atom of the pyridine, phenyl, or pyridine oxide (1-oxypyridin) ring:

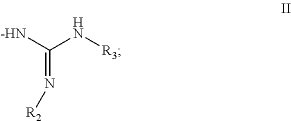

R₁ is 1, 2, 3, 4, or 5 substituents each independently selected from halogen, —COR₄, —COOR₄, —CON(R₄)₂, —OCOOR₄, or —OCON(R₄)₂;

R₂ is selected from H, —OH, —O—(C₁-C₈)alkyl, —CO—(C₁-C₈)alkyl, —COO—(C₁-C₅)alkyl, —CN, —CONH₂, or —NH₂;

R₃ is selected from (C₁-C₁₂)alkyl, (C₃-C₁₀)cycloalkyl, 3-7 membered heterocyclyl, (C₆-C₁₀)aryl, or 6-10-membered heteroaryl, optionally substituted with (C₆-C₁₀)aryl, or 6-10-membered heteroaryl;

R₄ each independently is selected from H, (C₃-C₁₀)cycloalkyl, 3-12-membered heterocyclyl, (C₆-C₁₄)aryl, or (C₁-C₈)alkyl optionally substituted with one or more groups each independently selected from —OR₅, —OSO₃⁻ (or a salt thereof such as —OSO₂(ONa)), —OPO₃²⁻ (or a salt thereof such as —OPO(ONa)₂), —OCF₃, —CF₃, —OCOR₅, —COR₅, —COOR₅, —OCOOR₅, —OCON(R₅)₂, —(C₁-C₅)alkylene-COOR₅, —CN, —NO₂, —ONO₂, —SRS, —N(R₅)₂, —CON(R₅)₂, —SO₂R₅, —S(═O)R₅, or glucuronic acid linked via the carboxyl group or one of the hydroxyl groups (e.g., the hydroxyl group at position C1) thereof; and

R₅ each independently is selected from H, (C₁-C₈)alkyl, (C₂-C₈)alkenyl, or (C₂-C₈)alkynyl.

In another aspect, the present invention provides a pharmaceutical composition comprising a compound of the formula I as defined above, or an enantiomer, diastereomer, racemate, or pharmaceutically acceptable salt thereof, and a pharmaceutically acceptable carrier. The compounds and pharmaceutical compositions of the invention are useful for prevention, treatment, or management of pulmonary arterial or venous hypertension.

In yet another aspect, the present invention relates to a compound of the formula I as defined above, or an enantiomer, diastereomer, racemate, or pharmaceutically acceptable salt thereof, for use in prevention, treatment, or management of pulmonary arterial or venous hypertension.

In still another aspect, the present invention relates to use of a compound of the formula I as defined above, or an enantiomer, diastereomer, racemate, or pharmaceutically acceptable salt thereof, for the preparation of a pharmaceutical composition for prevention, treatment, or management of pulmonary arterial or venous hypertension.

In a further aspect, the present invention relates to a method for prevention, treatment, or management of pulmonary arterial or venous hypertension, in a subject in need thereof, said method comprising administering to said subject a therapeutically effective amount of a compound of the formula I as defined above, or an enantiomer, diastereomer, racemate, or pharmaceutically acceptable salt thereof.

### BRIEF DESCRIPTION OF DRAWINGS

FIGS. 1A-1B show that IV-administered R-1703 produces a sustained reduction in MPAP in MCT-treated rats with PH. As shown, R-1703 dose-dependently reduced the MCT-induced elevation in MPAP, with evidence of complete restoration of normal pressures at a dose of 90 μg/kg (p<5×10⁻⁶) (1A); and the effect of R-1703 on MPAP persisted for at least 24 hours after administration (1B). Treatments with R-1703 had no effect on heart rate nor on peripheral blood pressure, indicating its absolute selectivity for the pulmonary vascular circulation at these dose levels.

FIG. 2 shows that intratracheal aerosolized R-1703 dose-dependently reduced the MCT-induced elevation in pulmonary arterial pressure (PAP, acute effect). Treatment with R-1703 had no effect on heart rate nor on peripheral blood pressure, thereby demonstrating the absolute selectivity for vasodilation of the pulmonary vascular circulation at these dose levels (p<<0.001).

FIG. 3 shows that intratracheal aerosolized R-1703 dose-dependently reduced the MCT-induced elevation in pulmonary arterial pressure (PAP, sustained effect). Treatment with R-1703 had no effect on heart rate nor on peripheral blood pressure, thereby demonstrating the absolute selectivity for vasodilation of the pulmonary vascular circulation at these dose levels (p<<0.001).

FIG. 4 shows that intraperitoneal delivery of R-1703 dose-dependently reduced the MCT-induced elevation in MPAP, with evidence of 80% restoration of normal pressure (p<<0.001).

### DETAILED DESCRIPTION

In one aspect, the present invention provides a compound of the general formula I as defined above, or an enantiomer, diastereomer, racemate, or a pharmaceutically acceptable salt thereof.

The term “halogen” as used herein includes fluoro, chloro, bromo, and iodo, and is preferably fluoro, or chloro.

The term “alkyl” as used herein typically means a linear or branched saturated hydrocarbyl having 1-12 carbon atoms and includes, e.g., methyl, ethyl, n-propyl, isopropyl, n-butyl, sec-butyl, isobutyl, tert-butyl, n-pentyl, isopentyl, neopentyl, 2,2-dimethylpropyl, n-hexyl, isohexyl, n-heptyl, 1,1-dimethylpentyl, 1,1-dimethylbutyl, 2,2-dimethylbutyl, 2-ethylbutyl, 1,1-dimethylheptyl, 1,2-dimethylheptyl, n-octyl, n-nonyl, n-decyl, n-undecyl, n-dodecyl, and the like. Preferred are (C₁-C₈)alkyl groups, more preferably methyl, ethyl, n-propyl, isopropyl, n-butyl, sec-butyl, isobutyl, or tert-butyl. The terms “alkenyl” and “alkynyl” typically mean linear or branched hydrocarbyls having 2-12, e.g., 2-8, carbon atoms and at least one double or triple bond, respectively, and include ethenyl, propenyl, 3-buten-1-yl, 2-ethenylbutyl, 3-octen-1-yl, 3-nonenyl, 3-decenyl, and the like, and propynyl, 2-butyn-1-yl, 3-pentyn-1-yl, 3-hexynyl, 3-octynyl, 4-decynyl, and the like. C₂-C₆ alkenyl and alkynyl groups are preferred, more preferably C₂-C₄ alkenyl and alkynyl. Each one of the alkyl, alkenyl, and alkynyl may be substituted, e.g., by one or more alkyl, aryl, or heteroaryl groups.

The term “alkylene” refers to a linear or branched divalent hydrocarbon radical having 1-12 carbon atoms, derived after removal of hydrogen atom from an alkyl, and includes, e.g., methylene, ethylene, propylene, butylene, 2-methylpropylene, pentylene, 2-methylbutylene, hexylene, 2-methylpentylene, 3-methylpentylene, 2,3-dimethylbutylene, heptylene, octylene, and the like. Preferred are (C₁-C₈)alkylene, e.g., (C₁-C₄)alkylene, more preferably methylene, ethylene or propylene.

The term “cycloalkyl” as used herein means a mono-, bi-, or poly-cyclic saturated hydrocarbyl having 3-10 carbon atoms such as cyclopropyl, cyclobutyl, cyclopentyl, cyclohexyl, cycloheptyl, cyclooctyl, adamantyl, bicyclo[3.2.1]octyl, bicyclo[2.2.1]heptyl, adamantly, and the like, that may be substituted, e.g., by one or more alkyl, aryl, or heteroaryl groups.

The term “heterocyclic ring” as used herein denotes a mono- or poly-cyclic non-aromatic ring of, e.g., 3-12 atoms containing at least two carbon atoms and at least one heteroatom selected from sulfur, oxygen, and nitrogen, which may be saturated or unsaturated, i.e., containing at least one unsaturated bond. Non-limiting examples of heterocyclic ring include pyrrolidine, piperidine, pyridine, dihydropyridine, tetrahydropyridine, pyrazole, pyrazoline, pyrazolidine, piperazine, imidazolidine, imidazoline, tetrahydropyrimidine, dihydrotriazine, azepane, ethylene oxide, furan, tetrahydrofuran, pyran, dihydropyran, tetrahydropyran, dioxole, dioxolane, morpholine, oxazolidine, oxazole, oxadiazole, oxazoline, dihydrooxadiazole, thiomorpholine, thiazolidine, thiazole, thiadiazole, and thiazoline. Preferred are 5- or 6-membered heterocyclic rings. The heterocyclic ring may be substituted at any of the ring atoms, e.g., by one or more alkyl, aryl, or heteroaryl groups. The term “heterocyclyl” as used herein refers to any univalent group derived from a heterocyclic ring as defined herein by removal of hydrogen atom from any ring atom.

The term “aryl” denotes an aromatic carbocyclic group having 6-14, e.g., 6-10, carbon atoms consisting of a single ring or multiple rings either condensed or linked by a covalent bond such as, but not limited to, phenyl, naphthyl, phenanthryl, and biphenyl. The aryl group may optionally be substituted by one or more groups each independently selected from halogen, (C₁-C₈)alkyl, —O—(C₁-C₈)alkyl, —COO(C₁-C₈)alkyl, —CN, —NO₂, aryl, or heteroaryl.

The term “heteroaryl” refers to a group derived from a mono- or poly-cyclic, e.g., 5-10-membered, heteroaromatic ring containing one to three, preferably 1-2, heteroatoms selected from N, O, or S. Examples of mono-cyclic heteroaryls include, without being limited to, pyrrolyl, furyl, thienyl, thiazinyl, pyrazolyl, pyrazinyl, imidazolyl, oxazolyl, isoxazolyl, thiazolyl, isothiazolyl, pyridyl, pyrimidinyl, 1,2,3-triazinyl, 1,3,4-triazinyl, and 1,3,5-triazinyl. Polycyclic heteroaryl groups are preferably composed of two rings such as, but not limited to, benzofuryl, isobenzofuryl, benzothienyl, indolyl, quinolinyl, isoquinolinyl, imidazo[1,2-a]pyridyl, benzimidazolyl, benzthiazolyl, benzoxazolyl, pyrido[1,2-a]pyrimidinyl and 1,3-benzodioxinyl. The heteroaryl may optionally be substituted by one or more groups each independently selected from halogen, (C₁-C₈)alkyl, —O—(C₁-C₈)alkyl, —COO(C₁-C₈)alkyl, —CN, —NO₂, aryl, or heteroaryl. It should be understood that when a polycyclic heteroaryl is substituted, the substitution may be in any of the carbocyclic and/or heterocyclic rings.

In certain embodiments, disclosed herein is a compound of the formula I, wherein Y is N; and A is linked to position 2, 3, or 4 of the pyridine ring, i.e., ortho, meta, or para to group Y (Table 1, formulae Ia₁, Ia₂ and Ia₃, respectively). In other embodiments, the invention provides a compound of the formula I, wherein Y is CH; and A is linked to any position of the phenyl ring (Table 1, formula Ib). In further embodiments, disclosed herein is a compound of the formula I, wherein Y is N(→O), and A is linked to position 2, 3, or 4 of the 1-oxypyridin ring, i.e., ortho, meta, or para to group Y (Table 1, formulae Ic₁, Ic₂ and Ic₃, respectively).

TABLE 1
The various formulae disclosed herein*

| | |
|---|---|
| 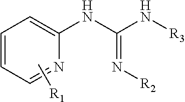 | Ia₁ |
| 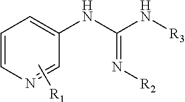 | Ia₂ |
| 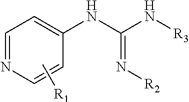 | Ia₃ |
| 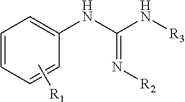 | Ib |
| 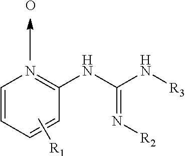 | Ic₁ |
| 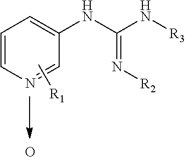 | Ic₂ |
| 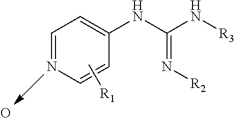 | Ic₃ |

*R₁, R₂ and R₃ are each as defined above.

According to the present invention, R₁ represents 1, 2, 3, 4, or 5 substituents as defined above. Yet, in cases wherein Y is N or N(→O), the maximal number of R₁ groups is limited to 4 only.

In certain embodiments, the present invention provides a compound of the formula I, i.e., a compound of the formula Ia₁, Ia₂, Ia₃, Ib, Ic₁, Ic₂, or Ic₃, wherein R₁ is 1, 2 or 3, preferably 1, substituents each independently selected from halogen, —COR₄, —COOR₄, —CON(R₄)₂, —OCOOR₄, or —OCON(R₄)₂; and R₄ each independently is H, or (C₁-C₈)alkyl, preferably (C₁-C₄)alkyl, optionally substituted with one or more groups each independently selected from —OR₅, —OSO₃⁻ (or a salt thereof such as —OSO₂(ONa)), —OPO₃²⁻ (or a salt thereof such as —OPO(ONa)₂), —COOR₅, —OCON(R₅)₂, —NO₂, —ONO₂, —N(R₅)₂, —CON(R₅)₂, or glucuronic acid linked via a hydroxyl or carboxyl group thereof. Particular such embodiments are those wherein R₁ represents 1-3 groups of the formula —CON(R₄)₂; and/or R₄ each independently is H, or (C₁-C₈)alkyl, preferably (C₁-C₄)alkyl, substituted with one or more groups each as defined above, e.g., wherein in each one of the —CON(R₄)₂ groups, one of R₄ is H, and the other one of R₄ is —CH₂OR₅, —CH₂CH₂OR₅, —CH₂CH₂CH₂OR₅, or —CH₂CH₂CH₂CH₂OR₅, wherein R₅ is H.

In certain embodiments, the present invention provides a compound of the formula I, i.e., a compound of the formula Ia₁, Ia₂, Ia₃, Ib, Ic₁, Ic₂, or Ic₃, wherein R₂ is CN.

In certain embodiments, the present invention provides a compound of the formula I, i.e., a compound of the formula Ia₁, Ia₂, Ia₃, Ib, Ic₁, Ic₂, or Ic₃, wherein R₃ is (C₁-C₁₂)alkyl, preferably (C₁-C₈)alkyl, optionally substituted with (C₆-C₁₀)aryl or 6-10-membered heteroaryl, or (C₃-C₁₀)cycloalkyl. In particular such embodiments, R₃ is a branched (C₄-C₅)alkyl such as, without limiting, 2-methylpropyl-1-yl, 2-methylpropyl-2-yl, 2-methylbutyl-1-yl, 2-methylbutyl-2-yl, 3-methylbutyl-2-yl, 2-methylpentyl-1-yl, 2-methylpentyl-2-yl, 2-methylpentyl-3-yl, 2-methylpentyl-4-yl, 2-methylpentyl-5-yl, 3-methylpentyl-1-yl, 3-methylpentyl-2-yl, 3-methylpentyl-3-yl, 2,3-dimethylbutyl-1-yl, 2,3-dimethylbutyl-2-yl, 2-methylhexyl-1-yl, 2-methylhexyl-2-yl, 2-methylhexyl-3-yl, 2-methylhexyl-4-yl, 2-methylhexyl-5-yl, 2-methylhexyl-6-yl, 3-methylhexyl-1-yl, 3-methylhexyl-2-yl, 3-methylhexyl-3-yl, 3-methylhexyl-4-yl, 3-methylhexyl-5-yl, 3-methylhexyl-6-yl, 2,2-dimethylpentyl-1-yl, 2,2-dimethylpentyl-3-yl, 2,2-dimethylpentyl-4-yl, 2,2-dimethylpentyl-5-yl, 3,3-dimethylpentyl-1-yl, 3,3-dimethylpentyl-2-yl, 2,3-dimethylpentyl-1-yl, 2,3-dimethylpentyl-2-yl, 2,3-dimethylpentyl-3-yl, 2,3-dimethylpentyl-4-yl, 2,3-dimethylpentyl-5-yl, 2,4-dimethylpentyl-1-yl, 2,4-dimethylpentyl-2-yl, 2,4-dimethylpentyl-3-yl, 2,4-dimethylpentyl-4-yl, 2,4-dimethylpentyl-5-yl, 2-methylheptyl-1-yl, 2-methylheptyl-2-yl, 2-methylheptyl-3-yl, 2-methylheptyl-4-yl, 2-methylheptyl-5-yl, 2-methylheptyl-6-yl, 2-methylheptyl-7-yl, 3-methylheptyl-1-yl, 3-methylheptyl-2-yl, 3-methylheptyl-3-yl, 3-methylheptyl-4-yl, 3-methylheptyl-5-yl, 3-methylheptyl-6-yl, 3-methylheptyl-7-yl, 4-methylheptyl-1-yl, 4-methylheptyl-2-yl, 4-methylheptyl-3-yl, 4-methylheptyl-4-yl, 2,2-dimethylhexyl-1-yl, 2,2-dimethylhexyl-3-yl, 2,2-dimethylhexyl-4-yl, 2,2-dimethylhexyl-5-yl, 2,2-dimethylhexyl-6-yl, 3,3-dimethylhexyl-1-yl, 3,3-dimethylhexyl-2-yl, 3,3-dimethylhexyl-4-yl, 3,3-dimethylhexyl-5-yl, 3,3-dimethylhexyl-6-yl, 2,3-dimethylhexyl-1-yl, 2,3-dimethylhexyl-2-yl, 2,3-dimethylhexyl-2-yl, 2,3-dimethylhexyl-4-yl, 2,3-dimethylhexyl-5-yl, 2,3-dimethylhexyl-6-yl, 2,4-dimethylhexyl-1-yl, 2,4-dimethylhexyl-2-yl, 2,4-dimethylhexyl-3-yl, 2,4-dimethylhexyl-4-yl, 2,4-dimethylhexyl-5-yl, 2,4-dimethylhexyl-6-yl, 2,5-dimethylhexyl-1-yl, 2,5-dimethylhexyl-2-yl, 2,5-dimethylhexyl-3-yl, 2,3,4-trimethylpentyl-1-yl, 2,3,4-trimethylpentyl-2-yl, and 2,3,4-trimethylpentyl-3-yl.

In certain embodiments, the present invention provides a compound of the formula I, i.e., a compound of the formula Ia₁, Ia₂, Ia₃, Ib, Ic₁, Ic₂, or Ic₃, wherein R₅ each independently is H, or (C₁-C₈)alkyl.

In certain embodiments, the present invention provides a compound of the formula I, more specifically a compound of the formula Ia₁, Ia₂, or Ia₃, wherein Y is N; A is linked to position 2, 3 or 4 of the pyridine ring; R₁ is 1, 2 or 3, preferably 1, substituents each independently selected from halogen, —COR₄, —COOR₄, —CON(R₄)₂, —OCOOR₄, or —OCON(R₄)₂; R₄ each independently is H, or (C₁-C₈)alkyl, preferably (C₁-C₄)alkyl, optionally substituted with one or more groups each independently selected from —OR₅, —OR₅, —OSO₃⁻ (or a salt thereof), —OPO₃²⁻ (or a salt thereof), —COOR₅, —OCON(R₅)₂, —NO₂, —ONO₂, —N(R₅)₂, —CON(R₅)₂, or glucuronic acid linked via a hydroxyl or carboxyl group thereof; R₂ is —CN; R₃ is (C₁-C₁₂)alkyl, preferably (C₁-C₈)alkyl, optionally substituted with (C₆-C₁₀)aryl or 6-10-membered heteroaryl, or (C₃-C₁₀)cycloalkyl; and R₅ each independently is H, or (C₁-C₈)alkyl. Particular such embodiments are those wherein R₁ represents 1-3 groups of the formula —CON(R₄)₂; and/or R₄ each independently is H, or (C₁-C₈)alkyl, preferably (C₁-C₄)alkyl, substituted with one or more groups each as defined above, e.g., wherein in each one of the —CON(R₄)₂ groups, one of R₄ is H, and the other one of R₄ is —CH₂OR₅, —CH₂CH₂OR₅, —CH₂CH₂CH₂OR₅, or —CH₂CH₂CH₂CH₂OR₅, wherein R₅ is H.

In particular embodiments as defined hereinabove, the invention provides a compound of the formula I, wherein Y is N; A is linked to position 2, 3 or 4 of the pyridine ring; R₁ represents a sole —CON(R₄)₂ group, wherein one of R₄ is H, and the other one of R₄ is —CH₂OH, —CH₂CH₂OH, —CH₂CH₂CH₂OH, or —CH₂CH₂CH₂CH₂OH; R₂ is —CN; and R₃ is a branched (C₄-C₈)alkyl as defined above, e.g., 2-methylbutyl-2-yl. In more particular such embodiments: (i) A is linked to position 2 of the pyridine ring; and R₁ is linked to position 3, 4, 5, or 6 of the pyridine ring, i.e., ortho, meta, or para to group A; (ii) A is linked to position 3 of the pyridine ring; and R₁ is linked to position 2, 4, 5, or 6 of the pyridine ring, i.e., ortho, meta, or para to group A; or (iii) A is linked to position 4 of the pyridine ring; and R₁ is linked to position 2 or 3 of the pyridine ring, i.e., ortho or meta to group A. In certain specific such embodiments, disclosed herein is a compound of the formula I, wherein Y is N; R₁ represents a sole —CON(R₄)₂ group, wherein one of R₄ is H, and the other one of R₄ is —CH₂CH₂OH; R₂ is —CN; R₃ is 2-methylbutyl-2-yl; and: (i) A is linked to position 2 of the pyridine ring, and R₁ is linked to position 3, 4, 5, or 6 of the pyridine ring (herein identified compounds 101, 102, 103, and 104, respectively); (ii) A is linked to position 3 of the pyridine ring, and R₁ is linked to position 2, 4, 5, or 6 of the pyridine ring (herein identified compounds 105, 106, 107 [referred to herein as R-1703], and 108, respectively); or (iii) A is linked to position 4 of the pyridine ring, and R₁ is linked to position 2 or 3 of the pyridine ring (herein identified compounds 109 and 110, respectively), or an enantiomer, diastereomer, racemate, or a pharmaceutically acceptable salt thereof. In a preferred embodiment exemplified herein, the compound disclosed is R 1703 (Table 2), or an enantiomer, diastereomer, racemate, or a pharmaceutically acceptable salt thereof.

In certain embodiments, the present invention provides a compound of the formula I, more specifically a compound of the formula Ib, wherein Y is CH; A is linked to any position of the phenyl ring; R₁ is 1, 2 or 3, preferably 1, substituents each independently selected from halogen, —COR₄, —COOR₄, —CON(R₄)₂, —OCOOR₄, or —OCON(R₄)₂; R₄ each independently is H, or (C₁-C₈)alkyl, preferably (C₁-C₄)alkyl, optionally substituted with one or more groups each independently selected from —OR₅, —OR₅, —OSO₃⁻ (or a salt thereof), —OPO₃²⁻ (or a salt thereof), —COOR₅, —OCON(R₅)₂, —NO₂, —ONO₂, —N(R₅)₂, —CON(R₅)₂, or glucuronic acid linked via a hydroxyl or carboxyl group thereof; R₂ is —CN; R₃ is (C₁-C₁₂)alkyl, preferably (C₁-C₈)alkyl, optionally substituted with (C₆-C₁₀)aryl or 6-10-membered heteroaryl, or (C₃-C₁₀)cycloalkyl; and R₅ each independently is H, or (C₁-C₈)alkyl. Particular such embodiments are those wherein R₁ represents 1-3 groups of the formula —CON(R₄)₂; and/or R₄ each independently is H, or (C₁-C₈)alkyl, preferably (C₁-C₄)alkyl, substituted with one or more groups each as defined above, e.g., wherein in each one of the —CON(R₄)₂ groups, one of R₄ is H, and the other one of R₄ is —CH₂OR₅, —CH₂CH₂OR₅, —CH₂CH₂CH₂OR₅, or —CH₂CH₂CH₂CH₂OR₅, wherein R₅ is H.

In particular embodiments as defined hereinabove, the invention provides a compound of the formula I, wherein Y is CH; A is linked to any position of the phenyl ring; R₁ represents a sole —CON(R₄)₂ group, wherein one of R₄ is H, and the other one of R₄ is —CH₂OH, —CH₂CH₂OH, —CH₂CH₂CH₂OH, or —CH₂CH₂CH₂CH₂OH; R₂ is —CN; and R₃ is a branched (C₄-C₈)alkyl as defined above, e.g., 2-methylbutyl-2-yl. In more particular such embodiments, R₁ is linked ortho, meta, or para to group A.

TABLE 2
Compounds of the formula Ia₁, Ia₂ or Ia₃ specifically disclosed herein

| | |
|---|---|
| 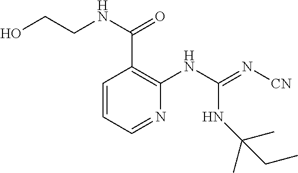 [SMILES: CCC(C)(C)N/C(=N\C#N)Nc1ncccc1C(=O)NCCO] | 101 |
| 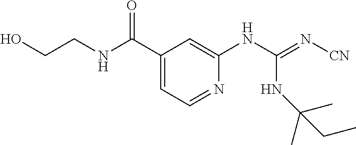 [SMILES: CCC(C)(C)N/C(=N\C#N)Nc1cc(C(=O)NCCO)ccn1] | 102 |
| 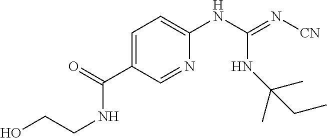 [SMILES: CCC(C)(C)N/C(=N\C#N)Nc1ccc(C(=O)NCCO)cn1] | 103 |
| 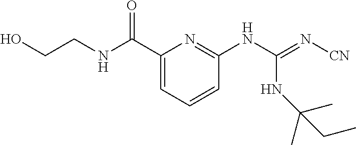 [SMILES: CCC(C)(C)N/C(=N\C#N)Nc1cccc(C(=O)NCCO)n1] | 104 |
| 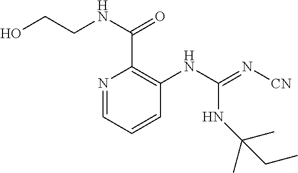 [SMILES: CCC(C)(C)N/C(=N\C#N)Nc1cccnc1C(=O)NCCO] | 105 |
| 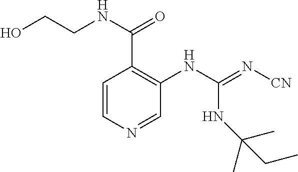 [SMILES: CCC(C)(C)N/C(=N\C#N)Nc1cnccc1C(=O)NCCO] | 106 |
| 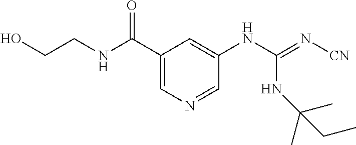 [SMILES: CCC(C)(C)N/C(=N\C#N)Nc1cncc(C(=O)NCCO)c1] | 107 |
| 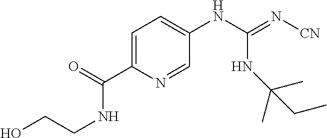 [SMILES: CCC(C)(C)N/C(=N\C#N)Nc1ccc(C(=O)NCCO)nc1] | 108 |
| 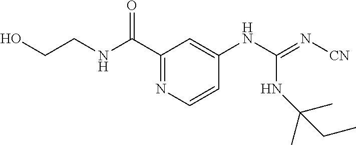 [SMILES: CCC(C)(C)N/C(=N\C#N)Nc1ccnc(C(=O)NCCO)c1] | 109 |
| 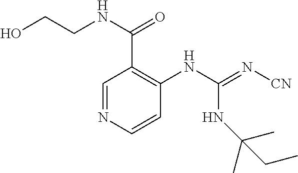 [SMILES: CCC(C)(C)N/C(=N\C#N)Nc1ccncc1C(=O)NCCO] | 110 |

In certain specific such embodiments, disclosed herein is a compound of the formula I, wherein Y is CH; R₁ represents a sole —CON(R₄)₂ group linked ortho, meta, or para to group A, wherein one of R₄ is H, and the other one of R₄ is —CH₂CH₂OH; R₂ is —CN; and R₃ is 2-methylbutyl-2-yl (herein identified compounds 111, 112 and 113, respectively) (Table 3), or an enantiomer, diastereomer, racemate, or a pharmaceutically acceptable salt thereof.

TABLE 3
Compounds of the formula Ib specifically disclosed herein

| | |
|---|---|
| 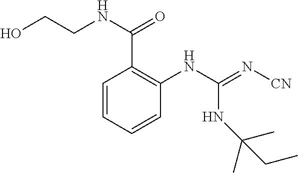 [SMILES: CCC(C)(C)N/C(=N\C#N)Nc1ccccc1C(=O)NCCO] | 111 |
| 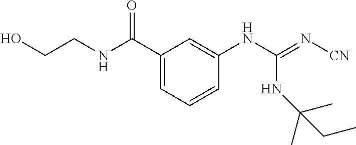 [SMILES: CCC(C)(C)N/C(=N\C#N)Nc1cccc(C(=O)NCCO)c1] | 112 |
| 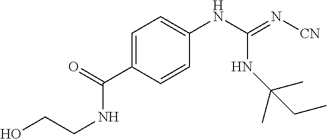 [SMILES: CCC(C)(C)N/C(=N\C#N)Nc1ccc(C(=O)NCCO)cc1] | 113 |

In certain embodiments, the present invention provides a compound of the formula I, more specifically a compound of the formula Ic₁, Ic₂, or Ic₃, wherein Y is N(→O); A is linked to position 2, 3 or 4 of the pyridine oxide ring; R₁ is 1, 2 or 3, preferably 1, substituents each independently selected from halogen, —COR₄, —COOR₄, —CON(R₄)₂, —OCOOR₄, or —OCON(R₄)₂; R₄ each independently is H, or (C₁-C₈)alkyl, preferably (C₁-C₄)alkyl, optionally substituted with one or more groups each independently selected from —OR₅, —OSO₃⁻ (or a salt thereof), —OPO₃²⁻ (or a salt thereof), —COOR₅, —OCON(R₅)₂, —NO₂, —ONO₂, —N(R₅)₂, —CON(R₅)₂, or glucuronic acid linked via a hydroxyl or carboxyl group thereof; R₂ is —CN; R₃ is (C₁-C₁₂)alkyl, preferably (C₁-C₈)alkyl, optionally substituted with (C₆-C₁₀)aryl or 6-10-membered heteroaryl, or (C₃-C₁₀)cycloalkyl; and R₅ each independently is H, or (C₁-C₈)alkyl. Particular such embodiments are those wherein R₁ represents 1-3 groups of the formula —CON(R₄)₂; and/or R₄ each independently is H, or (C₁-C₈)alkyl, preferably (C₁-C₄)alkyl, substituted with one or more groups each as defined above, e.g., wherein in each one of the —CON(R₄)₂ groups, one of R₄ is H, and the other one of R₄ is —CH₂OR₅, —CH₂CH₂OR₅, —CH₂CH₂CH₂OR₅, or —CH₂CH₂CH₂CH₂OR₅, wherein R₅ is H.

In particular embodiments as defined hereinabove, the invention provides a compound of the formula I, wherein Y is N(→O); A is linked to position 2, 3 or 4 of the pyridine oxide ring; R₁ represents a sole —CON(R₄)₂ group, wherein one of R₄ is H, and the other one of R₄ is —CH₂OH, —CH₂CH₂OH, —CH₂CH₂CH₂OH, or —CH₂CH₂CH₂CH₂OH; R₂ is —CN; and R₃ is a branched (C₄-C₈)alkyl as defined above, e.g., 2-methylbutyl-2-yl. In more particular such embodiments: (i) A is linked to position 2 of the pyridine oxide ring; and R₁ is linked to position 3, 4, 5, or 6 of the pyridine oxide ring, i.e., ortho, meta, or para to group A; (ii) A is linked to position 3 of the pyridine oxide ring; and R₁ is linked to position 2, 4, 5, or 6 of the pyridine oxide ring, i.e., ortho, meta, or para to group A; or (iii) A is linked to position 4 of the pyridine oxide ring; and R₁ is linked to position 2 or 3 of the pyridine oxide ring, i.e., ortho or meta to group A. In certain specific such embodiments, disclosed herein is a compound of the formula I, wherein Y is N(→O); R₁ represents a sole —CON(R₄)₂ group, wherein one of R₄ is H, and the other one of R₄ is —CH₂CH₂OH; R₂ is —CN; R₃ is 2-methylbutyl-2-yl; and: (i) A is linked to position 2 of the pyridine oxide ring and R₁ is linked to position 3, 4, 5, or 6 of the pyridine ring (herein identified compounds 114, 115, 116, and 117, respectively); (ii) A is linked to position 3 of the pyridine ring and R₁ is linked to position 2, 4, 5, or 6 of the pyridine oxide ring (herein identified compounds 118, 119, 120, and 121, respectively); or (iii) A is linked to position 4 of the pyridine oxide ring and R₁ is linked to position 2 or 3 of the pyridine oxide ring (herein identified compounds 122 and 123, respectively) (Table 4).

The compounds of the present invention may be synthesized according to any suitable technology or procedure known in the art, e.g., as described in the Examples section hereinafter.

The compounds of the formula I may have one or more asymmetric centers, e.g., in certain cases wherein one or more of the R₄ groups is a substituted (C₁-C₈)alkyl as well as in either or both the —NH groups of the guanidino moiety, and may accordingly exist both as enantiomers, i.e., optical isomers (R, S, or racemate, wherein a certain enantiomer may have an optical purity of 90%, 95%, 99% or more) and as diastereoisomers. The present invention encompasses all such enantiomers, isomers and mixtures thereof, as well as pharmaceutically acceptable salts thereof.

Optically active forms of the compounds of the invention may be prepared using any method known in the art, e.g., by resolution of the racemic form by recrystallization techniques; by chiral synthesis; by extraction with chiral solvents; or by chromatographic separation using a chiral stationary phase. A non-limiting example of a method for obtaining optically active materials is transport across chiral membranes, i.e., a technique whereby a racemate is placed in contact with a thin membrane barrier, the concentration or pressure differential causes preferential transport across the membrane barrier, and separation occurs as a result of the non-racemic chiral nature of the membrane that allows only one enantiomer of the racemate to pass through. Chiral chromatography, including simulated moving bed chromatography, can also be used. A wide variety of chiral stationary phases are commercially available.

TABLE 4
Compounds of the formula Ic₁, Ic₂ or Ic₃ specifically disclosed herein

| | |
|---|---|
| 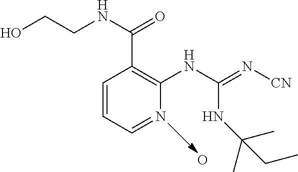 | 114 |
| 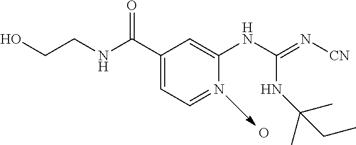 | 115 |
| 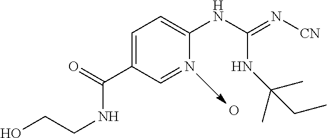 | 116 |
| 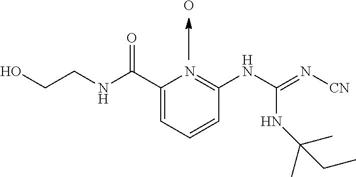 [SMILES: O ; CCC(C)(C)N/C(=N\C#N)Nc1cccc(C(=O)NCCO)n1] | 117 |
| 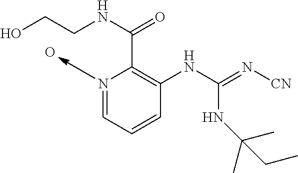 | 118 |
| 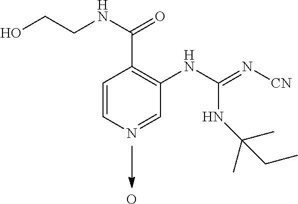 [SMILES: CCC(C)(C)N/C(=N\C#N)Nc1cnccc1C(=O)NCCO ; O] | 119 |
| 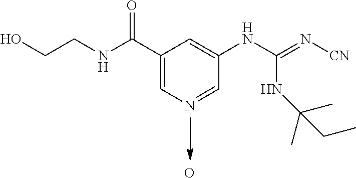 [SMILES: CCC(C)(C)N/C(=N\C#N)Nc1cncc(C(=O)NCCO)c1 ; O] | 120 |
| 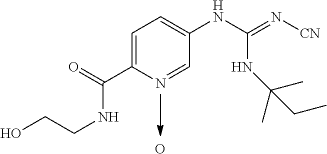 [SMILES: CCC(C)(C)N/C(=N\C#N)Nc1ccc(C(=O)NCCO)nc1 ; O] | 121 |
| 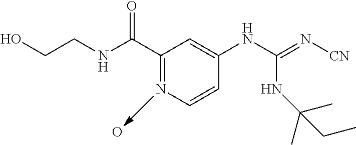 [SMILES: CCC(C)(C)N/C(=N\C#N)Nc1ccnc(C(=O)NCCO)c1 ; O] | 122 |
| 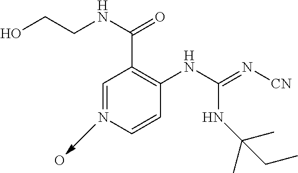 [SMILES: CCC(C)(C)N/C(=N\C#N)Nc1ccncc1C(=O)NCCO ; O] | 123 |

As shown herein, R-1703 was found to be highly effective in reducing the MCT-induced elevation in mean pulmonary arterial pressure, with evidence of complete restoration of normal pressures at a dose of 90 μg/kg, while maintaining its effect for at least 24 hours post dose; and to have no effect on heart rate nor on peripheral blood pressure, indicating its selectivity for the pulmonary vascular circulation at these dose levels. R-1703, as well as derivatives thereof (including enantiomers, diastereomers, racemates, and pharmaceutically acceptable salts thereof) as disclosed herein, are thus expected to be highly beneficial in treatment, prevention and/or managing of pulmonary arterial or venous hypertension, e.g., in prevention or management of acute life-threatening, perioperative PH in children with congenital heart defects (CHD) undergoing surgical correction of a left-to-right cardiac shunt.

In another aspect, the present invention thus provides a pharmaceutical composition comprising a compound of the formula I, i.e., a compound of the formula Ia₁, Ia₂, Ia₃, Ib, Ic₁, Ic₂, or Ic₃, as defined in any one of the embodiments above, or an enantiomer, diastereomer, racemate, or pharmaceutically acceptable salt thereof, herein also referred to as the active agent, and a pharmaceutically acceptable carrier. In certain embodiments, the active agent is any one of the compounds specifically shown in Tables 2-4 above, i.e., compounds 101-123, preferably R-1703, or an enantiomer, diastereomer, racemate, or pharmaceutically acceptable salt thereof.

The compounds and pharmaceutical compositions of the present invention are useful for prevention, treatment, or management of pulmonary hypertension, i.e., pulmonary arterial hypertension (PAH) or pulmonary venous hypertension (PVH).

The term “pulmonary hypertension” (PH) as used herein refers to a severe disease characterized by increased pulmonary vascular resistance, pulmonary arterial pressure, and ultimately pulmonary vascular remodeling effects that interfere with ventilation-perfusion relationships and compromise ventricular function. Several classification systems for PH have been published, including the Evian Nomenclature and Classification of PH (1998) and the Revised Nomenclature and Classification of PH (2003) (McCrory and Lewis, 2004).

PH may be either primary or secondary, and is currently classified into five groups, wherein PAH is classified as Group 1; PH associated with left heart diseases is classified as Group 2; PH associated with lung diseases and/or hypoxemia is classified as Group 3; PH due to chronic thrombotic and/or embolic diseases is classified as Group 4; and PH of other origin is classified as Group 5 (Galiè et al., 2004).

The term PAH as used herein refers to any PH including, without being limited to, idiopathic PAH (IPAH); familial PAH (FPAH); PAH associated with collagen vascular disease, e.g., scleroderma; PAH associated with heart disorders, e.g., congenital shunts between the systemic and pulmonary circulation, portal hypertension; PAH associated with HIV infection; PAH associated with venous or capillary diseases; PAH associated with thyroid disorders, glycogen storage disease, Gaucher's disease, hemoglobinopathies, or myeloproliferative disorders; PAH associated with either smoke inhalation or combined smoke inhalation and burn injury; PAH associated with aspiration; PAH associated with ventilator injury; PAH associated with pneumonia; PAH associated with Adult Respiratory Distress Syndrome; persistent PH of the newborn; neonatal respiratory distress syndrome of prematurity; neonatal meconium aspiration; neonatal diaphragmatic hernia; pulmonary capillary hemangiomatosis; and pulmonary veno-occlusive disease.

Examples of left heart disease that may be associated with Group 2 PH include, without limiting, left sided atrial or ventricular diseases, and valvular diseases, e.g., mitral stenosis.

Examples of lung diseases that may be associated with Group 3 PH include, without being limited to, chronic obstructive pulmonary disease (COPD), interstitial lung diseases (ILD), sleep-disordered breathing, alveolar hypoventilation disorders, chronic exposure to high altitude, and developmental lung abnormalities.

Examples of chronic thrombotic and/or embolic diseases that may be associated with Group 4 PH include, without limiting, thromboembolic obstruction of distal or proximal pulmonary arteries, and non-thrombotic pulmonary embolism of, e.g., tumor cells or parasites.

Examples of disorders or diseases that may be associated with Group 5 PH include, without being limited to, compression of pulmonary vessels by adenopathy, fibrosing mediastinitis, lymphangiomatosis, pulmonary Langerhans' cell granulomatosis (histiocytosis), sarcoidosis, hemoglobinopathy, and tumors.

Many of the diseases, disorders and conditions listed above can be associated with increased risk for PH, wherein particular examples, without limiting, include congenital heart disease, e.g., Eisenmenger syndrome; left heart disease; pulmonary venous disease, e.g., fibrosis tissue narrowing or occluding pulmonary veins and venules; pulmonary arterial disease; diseases causing alveolar hypoxia; fibrotic lung diseases; Williams syndrome; subjects with intravenous drug abuse injury; pulmonary vasculitis such as Wegener's, Goodpasture's, and Churg-Strauss syndromes; emphysema; chronic bronchitis; kyphoscoliosis; cystic fibrosis; obesity-hyper-ventilation and sleep apnea disorders; pulmonary fibrosis; sarcoidosis; silocosis; CREST (calcinosis cutis, Raynaud phenomenon; esophageal motility disorder; sclerodactyly, and teleangiectasia) and other connective tissue diseases. For example, a subject who possesses a bone morphogenetic protein receptor E (BMPR2) mutation has a 10-20% lifetime risk of acquiring FPAH, and subjects with hereditary hemorrhagic telangiectasa, particularly those carrying mutations in ALK1, were also identified as being at risk for IPAH. Risk factors and diagnostic criteria for PH are described in McGoon et al., 2004.

The compounds and pharmaceutical compositions of the present invention can be used for treatment any form of PH including, but not limited to, mild, i.e., associated with an increase of up to 30, more particularly 20-30, mmHg in MPAP at rest; moderate, i.e., associated with an increase of 30-39 mmHg in MPAP at rest; and severe, i.e., associated with an increase of 40 mmHg or more, e.g., of up to 60 mmHg, in MPAP at rest.

In certain embodiments, the compound of the present invention is used for the prevention and/or management of acute life-threatening PH in children with CHD undergoing correction of a left-to-right cardiac shunt. Pulmonary vascular disease is perhaps the most important complication for infants and children with CHD that result in increased pulmonary blood flow (PBF) and pressure, such as large ventricular septal defects (VSD) and atrioventricular septal defects (AVSD) (Hoffman et al., 1981; Burrows et al., 1986; Wheller et al., 1979; Rabinovitch et al., 1978). After birth, the presence of a systemic-to-pulmonary communication results in increasing PBF as PVR decreases. Exposure to this increased PBF and pressure leads to progressive structural and functional abnormalities of the pulmonary vascular bed (Rabinovitch et al., 1978). Without surgical repair, these abnormalities result in obliteration of the distal pulmonary arterioles and capillary bed and ultimately death secondary to right heart failure. With surgical correction, early vascular changes are reversible; however, more advanced changes are irreversible and progressive. Further, even children with reversible vascular changes suffer significant morbidity and mortality in the peri- and postoperative periods secondary to both chronic and acute elevations in PVR, particularly following exposure to cardiopulmonary bypass (CPB) (Hoffman et al., 1981; Gorenflo et al., 2010). For example, chronic elevations in PVR may result in diminished right ventricular function and low cardiac output, which has been associated with longer postoperative hospital length of stay, prolonged mechanical ventilation, and poor long-term outcomes (Brown et al., 2003; Schulze-Neick et al., 2001; Harrison et al., 2002). Acute elevations in PVR (i.e., pulmonary hypertensive crises) may result in sudden cardiopulmonary failure and cardiac arrest (Gorenflo et al., 2010). The incidence of these events appears to be decreasing with the trend toward early surgical repair and improved surgical techniques, but remains a substantial problem. For example, acute pulmonary hypertensive crises account for 8% of early (<30 days) mortality after repair of total anomalous pulmonary venous connection and remains a risk factor for late death (Bando et al., 1996; Cobanoglu and Menashe, 1993; Yong et al., 2011). In fact, estimates of overall mortality from acute pulmonary hypertensive crises have not decreased over the past decade, with a 20% mortality reported in 2010 (Fraisse et al., 2011; Lindberg et al., 2002; Loukanov et al., 2011). Moreover, the impact of pulmonary hypertensive crises in many regions outside the USA remains substantial, contributing to about 60% of postoperative deaths in India (Choudhary et al., 1999). Studies found that iNO in the post-operative period decreased the incidence of severe pulmonary hypertensive events and improved right ventricular function when used prophylactically (Checchia et al., 2012; Miller et al., 2000; Russell et al., 1998). However, the use of iNO is limited by an inconsistent and unpredictable response, excessive cost, and restricted availability. Thus, iNO is used more often to treat recognized pulmonary hypertension rather than for prevention (Checchia et al., 2012). Therefore, novel, efficacious, and cost-effective therapies for the prevention of pulmonary hypertensive crises are needed.

The term “treatment” as used herein with respect to PH refers to administration of an active agent as defined in any one of the embodiments above, after the onset of symptoms of PH in any of its forms. The term “prevention” as used herein with respect to PH refers to administration of the active agent prior to the onset of symptoms, particularly to patients at risk for PH; and the term “management” as used herein with respect to PH refers to prevention of recurrence of PH in a patient previously suffering from PH. The term “therapeutically effective amount” as used herein refers to the quantity of the active agent as defined above that is useful to treat, prevent, or manage the PH.

The term “subject” as used herein refers to a mammal, e.g., a human, non-human primate, horse, ferret, dog, cat, cow, and goat, but it preferably denotes a human, i.e., an individual.

The pharmaceutical compositions of the present invention can be provided in a variety of formulations, e.g., in a pharmaceutically acceptable form and/or a salt form, as well as in a variety of dosages.

In one embodiment, the pharmaceutical composition of the present invention comprises a non-toxic pharmaceutically acceptable salt of a compound of the formula I as defined above, or an enantiomer, diastereomer, racemate, or pharmaceutically acceptable salt thereof. Suitable pharmaceutically acceptable salts include acid addition salts such as, without being limited to, the mesylate salt, the maleate salt, the fumarate salt, the tartrate salt, the hydrochloride salt, the hydrobromide salt, the esylate salt, the p-toluenesulfonate salt, the benzenesulfonate salt, the benzoate salt, the acetate salt, the phosphate salt, the sulfate salt, the citrate salt, the carbonate salt, and the succinate salt. Additional pharmaceutically acceptable salts include salts of ammonium (NH₄⁺) or an organic cation derived from an amine of the formula R₄N⁺, wherein each one of the Rs independently is selected from H, C₁-C₂₂, preferably C₁-C₆ alkyl, such as methyl, ethyl, propyl, isopropyl, n-butyl, sec-butyl, isobutyl, tert-butyl, n-pentyl, 2,2-dimethylpropyl, n-hexyl, and the like, phenyl, or heteroaryl such as pyridyl, imidazolyl, pyrimidinyl, and the like, or two of the Rs together with the nitrogen atom to which they are attached form a 3-7 membered ring optionally containing a further heteroatom selected from N, S and O, such as pyrrolydine, piperidine and morpholine. Furthermore, where the compounds of the formula I carry an acidic moiety, suitable pharmaceutically acceptable salts thereof may include metal salts such as alkali metal salts, e.g., lithium, sodium and potassium salts, and alkaline earth metal salts, e.g., calcium and magnesium salts.

Further pharmaceutically acceptable salts include salts of a cationic lipid or a mixture of cationic lipids. Cationic lipids are often mixed with neutral lipids prior to use as delivery agents. Neutral lipids include, but are not limited to, lecithins; phosphatidylethanolamine; diacyl phosphatidylethanolamines such as dioleoyl phosphatidylethanolamine, dipalmitoyl phosphatidylethanolamine, palmitoyloleoyl phosphatidylethanolamine and distearoyl phosphatidylethanolamine; phosphatidylcholine; diacyl phosphatidylcholines such as dioleoyl phosphatidylcholine, dipalmitoyl phosphatidylcholine, palmitoyloleoyl phosphatidylcholine and distearoyl phosphatidylcholine; phosphatidylglycerol; diacyl phosphatidylglycerols such as dioleoyl phosphatidylglycerol, dipalmitoyl phosphatidylglycerol and distearoyl phosphatidylglycerol; phosphatidylserine; diacyl phosphatidylserines such as dioleoyl- or dipalmitoyl phosphatidylserine; and diphosphatidylglycerols; fatty acid esters; glycerol esters; sphingolipids; cardiolipin; cerebrosides; ceramides; and mixtures thereof. Neutral lipids also include cholesterol and other 3β hydroxy-sterols.

Examples of cationic lipid compounds include, without being limited to, Lipofectin® (Life Technologies, Burlington, Ontario) (1:1 (w/w) formulation of the cationic lipid N-[1-(2,3-dioleyloxy)propyl]-N,N,N-trimethylammonium chloride and dioleoylphosphatidyl-ethanolamine); Lipofectamine™ (Life Technologies, Burlington, Ontario) (3:1 (w/w) formulation of polycationic lipid 2,3-dioleyloxy-N-[2(spermine-carboxamido)ethyl]-N,N-dimethyl-1-propanamin-iumtrifluoroacetate and dioleoylphosphatidyl-ethanolamine), Lipofectamine Plus (Life Technologies, Burlington, Ontario) (Lipofectamine and Plus reagent), Lipofectamine 2000 (Life Technologies, Burlington, Ontario) (Cationic lipid), Effectene (Qiagen, Mississauga, Ontario) (Non liposomal lipid formulation), Metafectene (Biontex, Munich, Germany) (Polycationic lipid), Eu-fectins (Promega Biosciences, San Luis Obispo, Calif.) (ethanolic cationic lipids numbers 1 through 12: C₅₂H₁₀₆N₆O₄.4CF₃CO₂H, C₈₈H₁₇₈N₈O₄S₂.4CF₃CO₂H, C₄₀H₈₄NO₃P.CF₃CO₂H, C₅₀H₁₀₃N₇O₃.4CF₃CO₂H, C₅₅H₁₁₆N₈O₂.6CF₃CO₂H, C₄₉H₁₀₂N₆O₃.4CF₃CO₂H, C₄₄H₈₉N₅O₃.2CF₃CO₂H, C₁₀₀H₂₀₆N₁₂O₄S₂.8CF₃CO₂H, C₁₆₂H₃₃₀N₂₂O₉.13CF₃CO₂H, C₄₃H₈₈N₄O₂.2CF₃CO₂H, C₄₃H₈₈N₄O₃.2CF₃CO₂H, C₄₁H₇₈NO₈P); Cytofectene (Bio-Rad, Hercules, Calif.) (mixture of a cationic lipid and a neutral lipid), GenePORTER® (Gene Therapy Systems, San Diego, Calif.) (formulation of a neutral lipid (Dope) and a cationic lipid) and FuGENE 6 (Roche Molecular Biochemicals, Indianapolis, Ind.) (Multi-component lipid based non-liposomal reagent).

The pharmaceutically acceptable salts of the present invention may be formed by conventional means, e.g., by reacting a free base form of the compound of the formula I with one or more equivalents of the appropriate acid, in a solvent or medium in which the salt is insoluble, or in a solvent such as water which is removed in vacuo or by freeze drying; or by exchanging the anion/cation of an existing salt for another anion/cation on a suitable ion exchange resin.

Pharmaceutical compositions according to the present invention may be prepared by conventional techniques, e.g., as described in Remington: The Science and Practice of Pharmacy, 19^{th} Ed., 1995. The compositions can be prepared, e.g., by uniformly and intimately bringing the active agent into association with a liquid carrier, a finely divided solid carrier, or both, and then, if necessary, shaping the product into the desired formulation. The compositions may be in liquid, solid or semisolid form and may further include pharmaceutically acceptable fillers, carriers, diluents or adjuvants, and other inert ingredients and excipients. In one embodiment, the pharmaceutical composition of the present invention is formulated as nanoparticles.

The compositions can be formulated for any suitable route of administration, but they are preferably formulated for parenteral administration, e.g., for intravenous, intraarterial, intramuscular, intranasal, rectal, anal, intravaginal, intratracheal, intraperitoneal, or subcutaneous administration, as well as for inhalation. The dosage will depend on the state of the patient and will be determined as deemed appropriate by the practitioner.

The pharmaceutical composition of the invention may be in the form of a sterile injectable aqueous or oleagenous suspension, which may be formulated according to the known art using suitable dispersing, wetting or suspending agents. The sterile injectable preparation may also be a sterile injectable solution or suspension in a non-toxic parenterally acceptable diluent or solvent. Acceptable vehicles and solvents that may be employed include, without limiting, water, Ringer's solution, polyethylene glycol (PEG), 2-hydroxypropyl-β-cyclodextrin (HPCD), Tween-80, and isotonic sodium chloride solution.

Pharmaceutical compositions according to the present invention, when formulated for inhalation, may be administered utilizing any suitable device known in the art, such as metered dose inhalers, liquid nebulizers, dry powder inhalers, sprayers, thermal vaporizers, electrohydrodynamic aerosolizers, and the like.

In yet another aspect, the present invention relates to a compound of the formula I as defined in any one of the embodiments above, e.g., a compound selected from those specifically shown in Tables 2-4 above, preferably R-1703, or an enantiomer, diastereomer, racemate, or pharmaceutically acceptable salt thereof, for use in prevention, treatment or management of pulmonary arterial or venous hypertension, e.g., for use in prevention or management of acute life-threatening, perioperative PH in subjects (e.g., children) with CHD undergoing surgical correction of a left-to-right cardiac shunt.

In still another aspect, the present invention relates to use of a compound of the formula I as defined in any one of the embodiments above, e.g., a compound selected from those specifically shown in Tables 2-4 above, preferably R-1703, or an enantiomer, diastereomer, racemate, or pharmaceutically acceptable salt thereof, for the preparation of a pharmaceutical composition for prevention, treatment or management of pulmonary arterial or venous hypertension, e.g., for prevention or management of acute life-threatening, perioperative PH in subjects (e.g., children) with CHD undergoing surgical correction of a left-to-right cardiac shunt.

In a further aspect, the present invention relates to a method for prevention, treatment or management of pulmonary arterial or venous hypertension, in a subject in need thereof, comprising administering to said subject a therapeutically effective amount of a compound of the formula I as defined in any one of the embodiments above, e.g., a compound selected from those specifically shown in Tables 2-4 above, preferably R-1703, or an enantiomer, diastereomer, racemate, or pharmaceutically acceptable salt thereof. In certain embodiments, the method disclosed herein is for prevention or management of acute life-threatening, perioperative PH in subjects (e.g., children) with CHD undergoing surgical correction of a left-to-right cardiac shunt.

The invention will now be illustrated by the following non-limiting Examples.

### EXAMPLES

### Example 1. Synthesis of R-1703

As generally depicted in Scheme 1, the methyl ester 1 was first treated with aminoethanol to produce amide 2. Upon treatment with diphenyl N-cyano-carbonimidate and triethylamine in dichloromethane, compound 2 provided intermediate 3, which was then treated with 1,1-dimethylpropylamine to produce R-1703.

Step 1. 5-amino-nicotinic acid (10 g) was dissolved in methanol (80 ml). The solution was cooled to 0° C. and thionyl chloride (6.5 ml) was added dropwise. The resulting mixture was stirred at 0° C. for 30 minutes, and then at room temperature for 2 h. The reaction mixture was refluxed for 3 days. The reaction mixture was concentrated, and the residue was redissolved in 10% methanol in DCM. The solution was washed with a saturated sodium bicarbonate solution, brine and dried (Na₂SO₄). Solvent was removed under reduced pressure and the residue was recrystallized from DCM to give the desired product (9.4 g, 85%).

Step 2. Methyl 5-aminonicotinate (3.8 g) was suspended in 2-propanol (30 ml). Ethanolamine (5 ml) was added. The resulting mixture was heated at 90° C. for 3 days. The reaction mixture was concentrated, and the residue was recrystallized from 20% MeOH in DCM providing the desired amide (3.8 g, 84%).

Step 3. 5-amino-N-(2-hydroxyethyl)nicotinamide (1.2 g) and diphenyl cyanocarbonimidate (2.0 g) were dissolved in DMF (10 ml). The mixture was heated at 80° C. for 5 h and concentrated under reduced pressure. The residue was purified by column chromatography using 0-15% MeOH in DCM. Fraction was collected and concentrated to give the desired product 3 (892 mg, 43%).

Step 4. Compound 3 (560 mg) and amylamine (0.2 ml) were dissolved in DMF (5 ml). The mixture was heated at 70° C. for 3 days and concentrated under reduced pressure. The residue was purified by column chromatography using 0-15% MeOH in DCM. Impure fraction was collected and repurified to give the desired R-1703 (248 mg, 45%). ¹HNMR (DMSO-d6): 0.81 (t, J=8 Hz, 3H), 1.28 (s, 6H), 1.65 (m, 2H), 3.34 (m, 2H), 3.51 (m, 2H), 4.73 (m, 1H), 7.03 (s, 1H), 7.88 (s, 1H), 8.25 (s, 1H), 8.57 (s, 1H), 8.62 (s, 1H), 9.24 (s, 1H).

An alternative approach for the preparation of R-1703 is depicted in Scheme 2. According to this procedure, 5-aminonicotinic acid methyl ester 1 can be converted into intermediate 5 by treating with diphenyl N-cyano-carbonimidate. Introduction of the cyanoguanidine group is a crucial step in the synthesis. The intermediate 5 can be treated with 1,1-dimethylpropylamine to give intermediate 6. R-1703 is made by treating intermediate 6 with amino ethanol.

Further synthetic approaches for the preparation of R-1703, starting from 5-aminonicotinic acid methyl ester, are depicted in Schemes 3-4. The isocyanate intermediate 7 shown in Scheme 3 will be synthesized from compound 2 using carbon disulfide and triethylamine. It will be treated with 1,1-dimethylpropylamine in methylene chloride to produce the thiourea intermediate 8. The thiourea intermediate 8 can be converted to R-1703 by reacting with cyanamide in the presence of 1-ethyl-3-(3-dimethylaminopropyl)carbodiimide (EDAC or EDCI) and triethylamine.

R-1703 could also be synthesized from thiourea intermediate 8 as shown in Scheme 4. The thiourea 8 will be treated with copper sulphate and silica gel in THF, which will give the carbodiimide intermediate 9. The reactive carbodiimide group of intermediate 9 could react rapidly with cyanamide and produce R-1703 in two steps from thiourea. The conversion from intermediate 8 to R-1703 could also be performed in one step.

### Example 2. Acute and Sustained Effects R-1703 in a Rodent Treatment Model of MCT-Induced PH

An established rat model for PH utilizes monocrotaline (MCT), a toxin derived from plants of the Crotalaria species, which causes pulmonary arterial endothelial cell injury and subsequent pulmonary artery smooth muscle hypertrophy. This model serves as a tool for assessing an immediate vasorelaxation effect by monitoring real-time right ventricular pressure in rats with PAH (late intervention regimen), as well as assessment of efficacy in a prevention of vascular remodeling progression (early intervention regimen). In the latter mode, weight ratios of left and right ventricles and histopathological assessment of right ventricular hypertrophy are used to evaluate the extent of fibrosis.

R-1703 is effective in acutely treating a rodent model of MCT-induced PH. Sprague-Dawley rats (n=3 per group) were treated with a single subcutaneous (SC) injection of MCT with 60 mg/kg or an equivalent volume of saline (2 mL/kg). After a post-MCT period of 28 days wherein rats developed severe and stable PH, rats were anesthetized and endotracheally intubated, followed by a single intravenous (IV) administration of R-1703 (15, 30, 60, and 90 μg/kg, in PBS) via the tail vein. Vehicle controls received an equivalent volume of PBS.

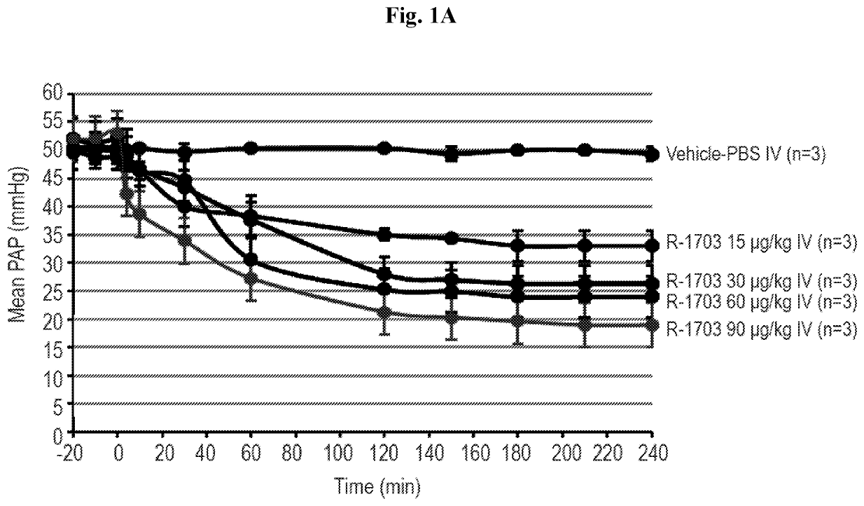

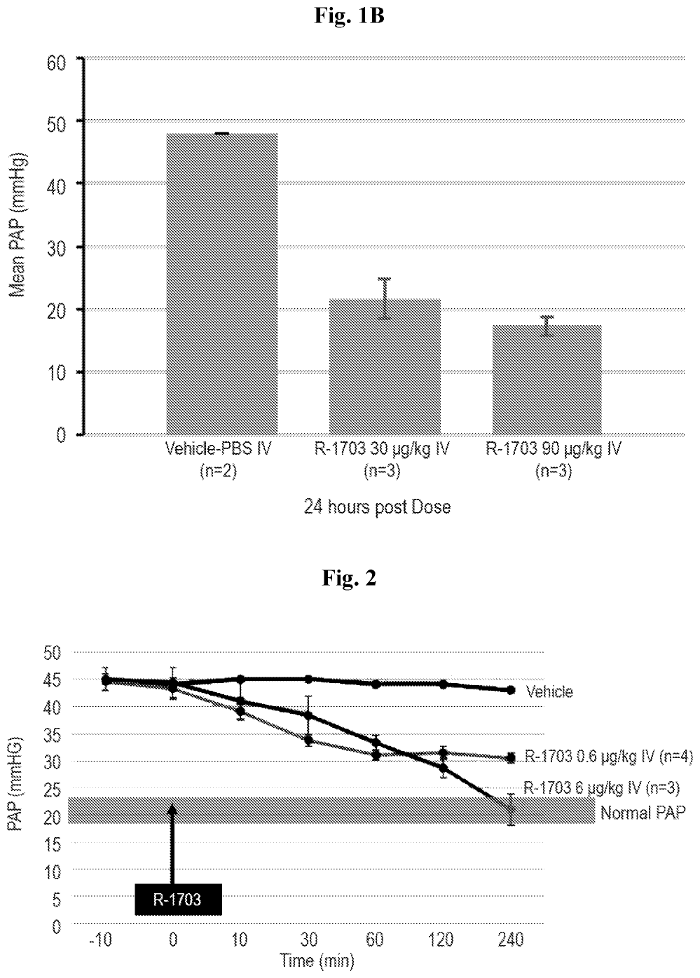

MCT induced a large increase in MPAP (from 25 to 50 mmHg). R-1703 dose-dependently reduced the MCT-induced elevation in MPAP, with evidence of complete (100%) restoration of normal pressures at a dose of 90 μg/kg (p<0.000005) (FIG. 1A). Treatment with R-1703 had no effect on heart rate nor on peripheral blood pressure, thereby demonstrating its absolute selectivity for vasodilation of the pulmonary vascular circulation at these dose levels. The effect of R-1703 on MPAP persisted for at least 24 h, post dose, thus exhibiting a prolonged duration of activity (FIG. 1B). The experiments shown below have been repeated three times and are reproducible.

Intratracheal (IT) aerosolized R-1703 is effective in a rodent treatment model of MCT-induced PH: Acute effect. 28 days after injection of MCT (60 mg/kg SC) and development of severe and stable PH, mechanically ventilated and anaesthetized Sprague-Dawley rats were dosed IT with aerosolized solution (2.0 mL) containing 500 or 5000 ng/mL of R-1703 in PBS. Vehicle controls received an equivalent volume of PBS. MCT induced a large increase in MPAP. IT R-1703 dose-dependently reduced the MCT-induced elevation in MPAP (p<0.001). Treatment with IT R-1703 had no effect on heart rate nor on peripheral blood pressure, thereby demonstrating the absolute selectivity for vasodilation of the pulmonary vascular circulation at these dose levels (FIG. 2).

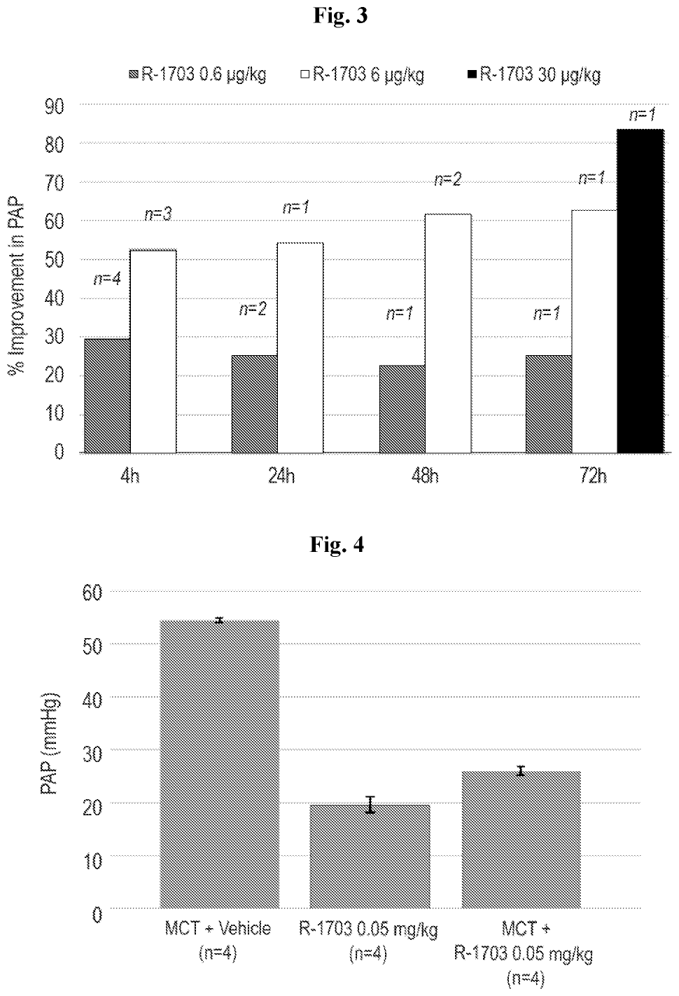

Intratracheal aerosolized R-1703 is effective in a rodent treatment model of MCT-induced PH: Sustained effect. 28 days after injection of MCT (60 mg/kg SC) and development of severe and stable PH, mechanically ventilated and anaesthetized Sprague-Dawley rats were dosed IT with aerosolized solution (2.0 mL) delivering 0.6, 6.0, or 30 micrograms per kg of R-1703 in PBS. Vehicle controls received an equivalent volume of PBS. MCT induced a large increase in MPAP. IT R-1703 dose-dependently reduced the MCT-induced elevation in MPAP (p<0.001). Treatment with IT R-1703 had no effect on heart rate nor on peripheral blood pressure, thereby demonstrating the absolute selectivity for vasodilation of the pulmonary vascular circulation at these dose levels (FIG. 3).

### Example 3. Prophylactic Therapy with R-1703 is Effective in a Rodent Treatment Model of MCT-Induced PH

Sprague-Dawley rats (n=3 per group) were treated with a single SC injection of MCT with 60 mg/kg or an equivalent volume of saline (2 mL/kg). Rats were then treated daily with 50 micrograms per kg of R-1703 by an intraperitoneal (IP) route of administration. After a post-MCT period of 28 days wherein vehicle control treated rats develop severe and stable PH, rats were anesthetized and endotracheally intubated. R-1703 dose-dependently was shown to have reduced the MCT-induced elevation in MPAP, with evidence of 80% restoration of normal pressures (p<0.001) (FIG. 4).

### APPENDIX

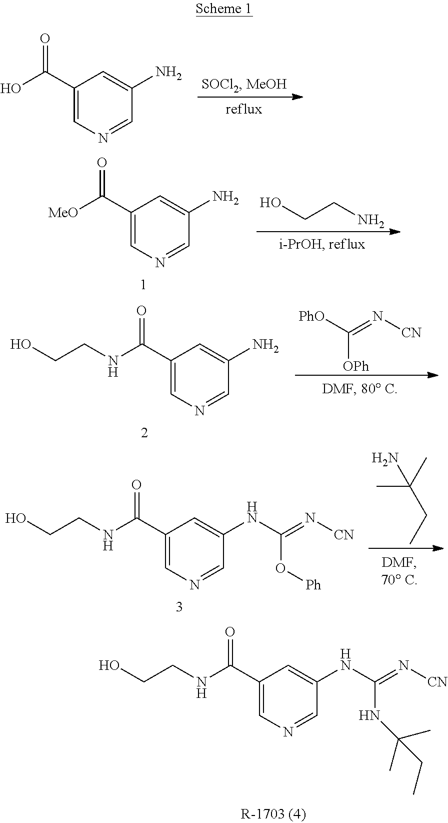

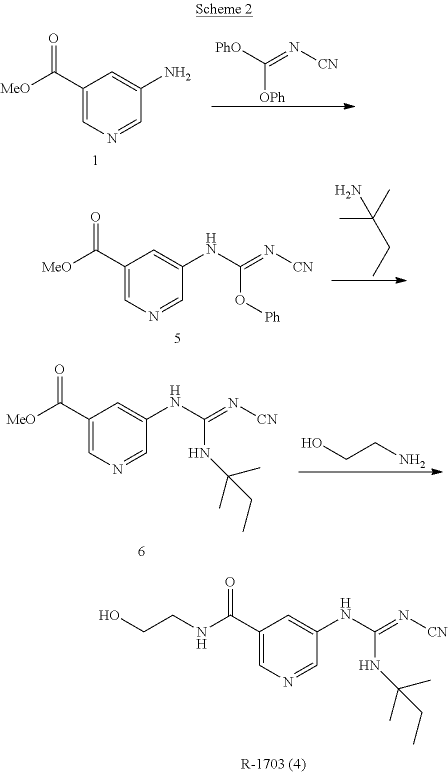

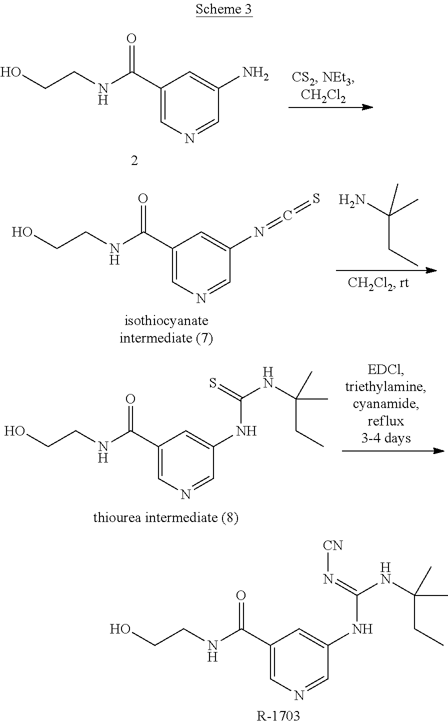

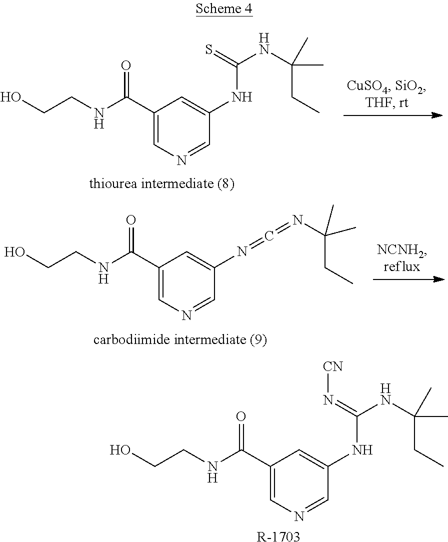

### REFERENCES

Bando, K.; Turrentine, M. W.; Ensing, G. J.; Sun, K.; Sharp, T. G.; Sekine, Y.; Girod, D. A.; Brown, J. W., Surgical management of total anomalous pulmonary venous connection. Thirty-year trends. Circulation 1996, 94, II12-6
Brown, K. L.; Ridout, D. A.; Goldman, A. P.; Hoskote, A.; Penny, D. J., Risk factors for long intensive care unit stay after cardiopulmonary bypass in children. Critical Care Medicine 2003, 31, 28-33
Burrows, F. A.; Klinck, J. R.; Rabinovitch, M.; Bohn, D. J., Pulmonary hypertension in children: perioperative management. Can Anaesth Soc J 1986, 33, 606-28
Checchia, P. A.; Bronicki, R. A.; Goldstein, B., Review of Inhaled Nitric Oxide in the Pediatric Cardiac Surgery Setting. Pediatric cardiology 2012
Choudhary, S. K.; Bhan, A.; Sharma, R.; Mathur, A.; Airan, B.; Saxena, A.; Kothari, S. S.; Juneja, R.; Venugopal, P., Repair of total anomalous pulmonary venous connection in infancy: experience from a developing country. The Annals of thoracic surgery 1999, 68, 155-9
Cobanoglu, A.; Menashe, V D., Total anomalous pulmonary venous connection in neonates and young infants: repair in the current era. The Annals of thoracic surgery 1993, 55, 43-8; discussion 48-9
Fraisse, A.; Butrous, G.; Taylor, M. B.; Oakes, M.; Dilleen, M.; Wessel, D. L., Intravenous sildenafil for postoperative pulmonary hypertension in children with congenital heart disease. Intensive care medicine 2011, 37, 502-9
Galiè, N.; Torbicki, A.; Barst, R.; Dartevelle, P.; Haworth, S.; Higenbottam, T.; Olschewski, H.; Peacock, A.; Pietra, G.; Rubin, L. J.; Simonneau, G.; Priori, S. G.; Garcia, M. A.; Blanc, J. J.; Budaj, A.; Cowie, M.; Dean, V.; Deckers, J.; Burgos, E. F.; Lekakis, J.; Lindahl, B.; Mazzotta, G.; McGregor, K.; Morais, J.; Oto, A.; Smiseth, O. A.; Barbera, J. A.; Gibbs, S.; Hoeper, M.; Humbert, M.; Naeije, R.; Pepke-Zaba, J., Guidelines on diagnosis and treatment of pulmonary arterial hypertension. The task force on diagnosis and treatment of pulmonary arterial hypertension of the European Society of Cardiology, Eur Heart J., 2004, 25, 2243-2278
Gorenflo, M.; Gu, H.; Xu, Z., Peri-operative pulmonary hypertension in paediatric patients: current strategies in children with congenital heart disease. Cardiology 2010, 116, 10-7
Harrison, A. M.; Cox, A. C.; Davis, S.; Piedmonte, M.; Drummond-Webb, J. J.; Mee, R. B., Failed extubation after cardiac surgery in young children: Prevalence, pathogenesis, and risk factors. Pediatric critical care medicine: a journal of the Society of Critical Care Medicine and the World Federation of Pediatric Intensive and Critical Care Societies 2002, 3, 148-152
Hoffman, J. I.; Rudolph, A. M.; Heymann, M. A., Pulmonary vascular disease with congenital heart lesions: pathologic features and causes. Circulation 1981, 64, 873-7
Hoffman, J. I.; Kaplan, S., The incidence of congenital heart disease. Journal of the American College of Cardiology 2002, 39, 1890-900
Lindberg, L.; Olsson, A. K.; Jogi, P.; Jonmarker, C., How common is severe pulmonary hypertension after pediatric cardiac surgery? The Journal of thoracic and cardiovascular surgery 2002, 123, 1155-63
Loukanov, T.; Bucsenez, D.; Springer, W.; Sebening, C.; Rauch, H.; Roesch, E.; Karck, M.; Gorenflo, M., Comparison of inhaled nitric oxide with aerosolized iloprost for treatment of pulmonary hypertension in children after cardiopulmonary bypass surgery. Clinical research in cardiology: official journal of the German Cardiac Society 2011, 100, 595-602
McCrory and Lewis, Methodology and grading for pulmonary hypertension evidence review and guideline development, Chest, 2004, 126, 11-13
McGoon, M.; Gutterman, D.; Steen, V.; Barst, R.; McCrory, D. C.; Fortin, T. A.; Loyd, J. E., Chest, 2004, 126, 14S-34S
Miller, O. I.; Tang, S. F.; Keech, A.; Pigott, N. B.; Beller, E.; Celermajer, D. S., Inhaled nitric oxide and prevention of pulmonary hypertension after congenital heart surgery: a randomised double-blind study. Lancet 2000, 356, 1464-9
Rabinovitch, M.; Haworth, S. G.; Castaneda, A. R.; Nadas, A. S.; Reid, L. M., Lung biopsy in congenital heart disease: a morphometric approach to pulmonary vascular disease. Circulation 1978, 58, 1107-22
Russell, I. A.; Zwass, M. S.; Fineman, J. R.; Balea, M.; Rouine-Rapp, K.; Brook, M.; Hanley, F. L.; Silverman, N. H.; Cahalan, M. K., The effects of inhaled nitric oxide on postoperative pulmonary hypertension in infants and children undergoing surgical repair of congenital heart disease. Anesth Analg 1998, 87, 46-51
Schulze-Neick, I.; Li, J.; Penny, D. J.; Redington, A. N., Pulmonary vascular resistance after cardiopulmonary bypass in infants: effect on postoperative recovery. The Journal of thoracic and cardiovascular surgery 2001, 121, 1033-9
Wheller, J.; George, B. L.; Mulder, D. G.; Jarmakani, J. M., Diagnosis and management of postoperative pulmonary hypertensive crisis. Circulation 1979, 60, 1640-4
Yong, M. S.; d'Udekem, Y.; Robertson, T.; Horton, S.; Dronavalli, M.; Brizard, C.; Weintraub, R.; Shann, F.; Cheung, M.; Konstantinov, I. E., Outcomes of surgery for simple total anomalous pulmonary venous drainage in neonates. The Annals of thoracic surgery 2011, 91, 1921-7

1. A compound of the general formula I: 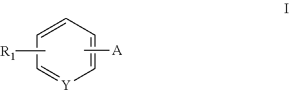
wherein Y is N, CH or N(→O),
or an enantiomer, diastereomer, racemate, or a pharmaceutically acceptable salt thereof,
wherein
A is a moiety of the formula II linked through its terminal —NH group to any carbon atom of the pyridine, phenyl, or pyridine oxide ring: 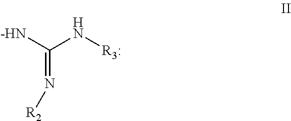
R₁ is 1, 2 or 3 substituents each independently of the formula —CON(R₄)₂;
R₂ is selected from the group consisting of H, —OH, —O—(C₁-C₈)alkyl, —CO—(C₁-C₈)alkyl, —COO—(C₁-C₈)alkyl, —CN, —CONH₂, and —NH₂;
R₃ is selected from the group consisting of (C₁-C₁₂)alkyl, (C₃-C₁₀)cycloalkyl, 3-7 membered heterocyclyl, (C₆-C₁₀)aryl, and 5-10-membered heteroaryl, optionally substituted with (C₆-C₁₀)aryl, or 6-10-membered heteroaryl;
one of R₄ is H, and the other one of R₄ is (C₁-C₈)alkyl optionally substituted with one or more groups each independently selected from the group consisting of —OR₅, —OSO₃⁻, —OPO₃²⁻, —OCF₃, —CF₃, —OCOR₅, —COR₅, —COOR₅, —OCOOR₅, —OCON(R₅)₂, —(C₁-C₈)alkylene-COOR₅, —CN, —NO₂, —ONO₂, —SR₅, —N(R₅)₂, —CON(R₅)₂, —SO₂R₅, —S(═O)R₅, and glucuronic acid linked via a hydroxyl or carboxyl group thereof; and
R₅ each independently is selected from the group consisting of H, (C₁-C₈)alkyl, (C₂-C₈)alkenyl, and (C₂-C₈)alkynyl.

2. The compound of claim 1, wherein (i) Y is N, and A is linked to position 2, 3, or 4 of the pyridine ring; (ii) Y is CH, and A is linked to any position of the phenyl ring; or (iii) Y is N(→O), and A is linked to position 2, 3 or 4 of the 1-oxypyridin ring.

3. (canceled)

4. The compound of claim 1, wherein R₁ is —CON(R₄)₂; one of R₄ is H; and the other one of R₄ is —CH₂OH, —(CH₂)₂—OH, —(CH₂)₃—OH, or —(CH₂)₄—OH.

5. The compound of claim 1, wherein R₂ is CN.

6. The compound of claim 1, wherein R₃ is (C₁-C₁₂)alkyl optionally substituted with (C₆-C₁₀)aryl or 6-10-membered heteroaryl, or (C₃-C₁₀)cycloalkyl.

7. The compound of claim 6, wherein R₃ is branched (C₄-C₈)alkyl.

8. The compound of claim 1, wherein R₅ each independently is H, or (C₁-C₈)alkyl.

9. The compound of claim 1, wherein Y is N; A is linked to position 2, 3 or 4 of the pyridine ring; R₁ is 1, 2 or 3 substituents each independently of the formula —CON(R₄)₂; one of R₄ is H, and the other one of R₄ is (C₁-C₈)alkyl optionally substituted with one or more groups each independently selected from the group consisting of —OR₅, —OSO₃⁻, —OPO₃²⁻, —COOR₅, —OCON(R₅)₂, —NO₂, —ONO₂, —N(R₅)₂, —CON(R₅)₂, and glucuronic acid linked via a hydroxyl or carboxyl group thereof; R₂ is —CN; R₃ is (C₁-C₁₂)alkyl optionally substituted with (C₆-C₁₀)aryl or 6-10-membered heteroaryl, or (C₃-C₁₀)cycloalkyl; and R₅ each independently is H, or (C₁-C₈)alkyl.

10. The compound of claim 9, wherein R₁ is —CON(R₄)₂; one of R₄ is H; the other one of R₄ is —CH₂OH, —(CH₂)₂—OH, —(CH₂)₃—OH, or —(CH₂)₄—OH; and R₃ is branched (C₄-C₈)alkyl.

11. The compound of claim 10, wherein R₃ is 2-methylbutyl-2-yl.

12. The compound of claim 10, wherein (i) A is linked to position 2 of the pyridine ring; and R₁ is linked ortho, meta, or para to group A; (ii) A is linked to position 3 of the pyridine ring; and R₁ is linked ortho, meta, or para to group A; or (iii) A is linked to position 4 of the pyridine ring; and R₁ is linked ortho or meta to group A.

13. The compound of claim 12, wherein one of R₄ is H; the other one of R₄ is —CH₂CH₂OH; R₃ is 2-methylbutyl-2-yl; and
(i) A is linked to position 2 of the pyridine ring; and R₁ is linked to position 3, 4, 5, or 6 of the pyridine ring, herein identified compounds 101, 102, 103, and 104, respectively;
(ii) A is linked to position 3 of the pyridine ring; and R₁ is linked to position 2, 4, 5, or 6 of the pyridine ring, herein identified compounds 105, 106, 107, and 108, respectively; or
(iii) A is linked to position 4 of the pyridine ring; and R₁ is linked to position 2 or 3 of the pyridine ring, herein identified compounds 109 and 110,
respectively.

14. The compound of claim 13, wherein A is linked to position 3 of the pyridine ring; and R₁ is linked to position 5 of the pyridine ring.

15. The compound of claim 1, wherein Y is CH; A is linked to any position of the phenyl ring; R₁ is 1, 2 or 3 substituents each independently of the formula —CON(R₄)₂; one of R₄ is H, and the other one of R₄ is (C₁-C₈)alkyl optionally substituted with one or more groups each independently selected from the group consisting of —OR₅, —OSO₃⁻, —OPO₃²⁻, —COOR₅, —OCON(R₅)₂, —NO₂, —ONO₂, —N(R₅)₂, —CON(R₅)₂, and glucuronic acid linked via a hydroxyl or carboxyl group thereof; R₂ is —CN; R₃ is (C₁-C₁₂)alkyl optionally substituted with (C₆-C₁₀)aryl or 6-10-membered heteroaryl, or (C₃-C₁₀)cycloalkyl; and R₅ each independently is H, or (C₁-C₈)alkyl.

16. The compound of claim 15, wherein R₁ is —CON(R₄)₂; one of R₄ is H; the other one of R₄ is —CH₂OH, —(CH₂)₂—OH, —(CH₂)₃—OH, or —(CH₂)₄—OH; and R₃ is branched (C₄-C₈)alkyl.

17. The compound of claim 16, wherein R₃ is 2-methylbutyl-2-yl.

18. The compound of claim 16, wherein R₁ is linked ortho, meta, or para to group A, herein identified compounds 111, 112 and 113, respectively.

19. The compound of claim 1, wherein Y is N(→O); A is linked to position 2, 3 or 4 of the pyridine oxide ring; R₁ is 1, 2 or 3 substituents each independently of the formula —CON(R₄)₂; one of R₄ is H, and the other one of R₄ is (C₁-C₈)alkyl optionally substituted with one or more groups each independently selected from the group consisting of —OR₅, —OSO₃, —OPO₃²⁻, —COOR₅, —OCON(R₅)₂, —NO₂, —ONO₂, —N(R₅)₂, —CON(R₅)₂, and glucuronic acid linked via a hydroxyl or carboxyl group thereof; R₂ is —CN; R₃ is (C₁-C₁₂)alkyl optionally substituted with (C₆-C₁₀)aryl or 6-10-membered heteroaryl, or (C₃-C₁₀)cycloalkyl; and R₅ each independently is H, or (C₁-C₈)alkyl.

20. The compound of claim 19, wherein R₁ is —CON(R₄)₂; one of R₄ is H; the other one of R₄ is —CH₂OH, —(CH₂)₂—OH, —(CH₂)₃—OH, or —(CH₂)₄—OH; and R₃ is branched (C₄-C₈)alkyl.

21. The compound of claim 20, wherein R₃ is 2-methylbutyl-2-yl.

22. The compound of claim 20 or 21, wherein:
(i) A is linked to position 2 of the pyridine oxide ring; and R₁ is linked ortho, meta, or para to group A, herein identified compounds 114, 115, 116, and
117, respectively;
(ii) A is linked to position 3 of the pyridine oxide ring; and R₁ is linked ortho, meta, or para to group A, herein identified compounds 118, 119, 120, and 121, respectively; or
(iii) A is linked to position 4 of the pyridine oxide ring; and R₁ is linked ortho or meta to group A, herein identified compounds 122 and 123, respectively.

23. A pharmaceutical composition comprising a compound according to claim 1, or an enantiomer, diastereomer, racemate, or pharmaceutically acceptable salt thereof, and a pharmaceutically acceptable carrier.

24. The pharmaceutical composition of claim 23, formulated for intravenous, intraarterial, intramuscular, intranasal, rectal, anal, intravaginal, intratracheal, intraperitoneal, or subcutaneous, administration, or for inhalation.

25-29. (canceled)

30. A method for prevention, treatment, or management of pulmonary hypertension (pulmonary arterial hypertension or pulmonary venous hypertension) in a subject in need thereof, comprising administering to said subject a therapeutically effective amount of a compound according to claim 1, or an enantiomer, diastereomer, racemate, or pharmaceutically acceptable salt thereof.

31. The method of claim 30, wherein said pulmonary hypertension is selected from the group consisting of pulmonary arterial hypertension, pulmonary hypertension associated with left heart diseases, pulmonary hypertension associated with a lung disease and/or hypoxemia, pulmonary hypertension due to chronic thrombotic and/or embolic disease, and pulmonary hypertension of other origin.

32. The method of claim 30, for prevention or management of acute life-threatening, perioperative pulmonary hypertension in a subject with a congenital heart defect undergoing surgical correction of a left-to-right cardiac shunt.
